SEQUENCE LISTING (21 sequences)

SEQ ID NO: 1 - DNA, 1980 bp, Aspergillus niger
atggccctcacatcctgggaacaaaccgcagcggccaaacgccaatccgtcctcaacgccatccccgagaaatggcgcatcaagggtcctatccccgcaccgtcggagcagcgcgacgtaacaggcccctacatccagcagttcctatccccacgcgaggttgaaatcaccgaaacagacgccgtagggatcacagagcgaactacaacgggccagtggacagctgtggaggtgaccgaggcgttctgccatcgcgcagcattggcgcatcaactcgtacattccccatccacaaggagtgctagtctgcgctttactaatcgagaaaaaggtaaactgcttgcatgaaatcttcttcgatgccgcgcttgaaaccgcccgcattctagacgaccactacaccaagaccggcaagccactcggtccccttcacggcctccctgtcagtctgaaggatcaattccacgtcaagggcgtagaaacaaccatgggttacgtcggctggataaacaccttccaaggcaagaccaatgacccgcgctatcttacacacgaaagcgaactcgttaaagaactccgcgccgcgggagccgtcctctactgcaagactagcgtccccatgacgttgatgtcaggtgaaaccatgaacaatatcataacttacacacataacccgaagaacaggcttctcagttctggaggtagttccgggggcgaaggagcactgatcgcgttgcggggatcaccagccgggtttggtacggatatcgggggtagtatccgtgttcctgcgtcgttcaatggactgtatgggatacggccgtctgtggggagaatgccgtacgagggggcggccaattcgggcgatggacagaatactgtgttgtcggttgtggggccgttgtctccttcggcgagagggttgatattgctgttcaagacggtgttgggggcaatgccgtggttgggagatcctggtgtgttggagattccctggagggaggaaatcgtagaggagacgagaaaattagtgcagggaaagccagaggggctagcttttggaatattctacgatgatggtcaggtaaagccgcagccaccggtcgagagagcgatgcggattgctgcagagacgatcaagcgtctaggacataaggtgagtgccctccttcttcttgcgacactgctaacattcatcccagctcatcaattgggaacccccctctcacctaacagccgcctccctcgcagtaagtcccccatccaacccactacaccacaaccccctaacaataaaccaacccccagaaccgcgcctacaacatggacggcggcgccgacgtactccaaaacttcgccctgtccaacgaagccatccacacctccgtagtaatcgacgcatcaggatccccccaaaagaccgcactagagatcgccgcgctaaacgtcgagaagcgcgaataccagaaacaataccttgactactggaacagcacggcgcaattgacagggactggacgacccgtcgacgcggtcatttgtccagtggcgccgcatgcggcgtgcattccggggaagtatgcgacgatcgggtatacggcgtttattaatgtgttggattatacgagtgcggttgtgccggttacgagtgctgataggagggtggatgttgtagggaaggaaggaagggagtattttggggagttggataggaagaccgagggggagtgtaagttcttccctttcttttcttctttcttttcattgagctatccaatttggttggaggtcttgtgtgtttgtttgttcggagagtggtgatggggttatgtgctgactggatgtttctatctagacgatgcggatgtgtttgatggggcgccggctgggattcagctctttggaagacggcttcaggaggagaagattctggtactggctgagtatcttggtgaggaattcaagaaggctagtgcttga

SEQ ID NO: 2 - DNA, 1695 bp, Aspergillus niger
atggccctcacatcctgggaacaaaccgcagcggccaaacgccaatccgtcctcaacgccatccccgagaaatggcgcatcaagggtcctatccccgcaccgtcggagcagcgcgacgtaacaggcccctacatccagcagttcctatccccacgcgaggttgaaatcaccgaaacagacgccgtagggatcacagagcgaactacaacgggccagtggacagctgtggaggtgaccgaggcgttctgccatcgcgcagcattggcgcatcaactcgtaaactgcttgcatgaaatcttcttcgatgccgcgcttgaaaccgcccgcattctagacgaccactacaccaagaccggcaagccactcggtccccttcacggcctccctgtcagtctgaaggatcaattccacgtcaagggcgtagaaacaaccatgggttacgtcggctggataaacaccttccaaggcaagaccaatgacccgcgctatcttacacacgaaagcgaactcgttaaagaactccgcgccgcgggagccgtcctctactgcaagactagcgtccccatgacgttgatgtcaggtgaaaccatgaacaatatcataacttacacacataacccgaagaacaggcttctcagttctggaggtagttccgggggcgaaggagcactgatcgcgttgcggggatcaccagccgggtttggtacggatatcgggggtagtatccgtgttcctgcgtcgttcaatggactgtatgggatacggccgtctgtggggagaatgccgtacgagggggcggccaattcgggcgatggacagaatactgtgttgtcggttgtggggccgttgtctccttcggcgagagggttgatattgctgttcaagacggtgttgggggcaatgccgtggttgggagatcctggtgtgttggagattccctggagggaggaaatcgtagaggagacgagaaaattagtgcagggaaagccagaggggctagcttttggaatattctacgatgatggtcaggtaaagccgcagccaccggtcgagagagcgatgcggattgctgcagagacgatcaagcgtctaggacataagctcatcaattgggaacccccctctcacctaacagccgcctccctcgcaaaccgcgcctacaacatggacggcggcgccgacgtactccaaaacttcgccctgtccaacgaagccatccacacctccgtagtaatcgacgcatcaggatccccccaaaagaccgcactagagatcgccgcgctaaacgtcgagaagcgcgaataccagaaacaataccttgactactggaacagcacggcgcaattgacagggactggacgacccgtcgacgcggtcatttgtccagtggcgccgcatgcggcgtgcattccggggaagtatgcgacgatcgggtatacggcgtttattaatgtgttggattatacgagtgcggttgtgccggttacgagtgctgataggagggtggatgttgtagggaaggaaggaagggagtattttggggagttggataggaagaccgagggggagtacgatgcggatgtgtttgatggggcgccggctgggattcagctctttggaagacggcttcaggaggagaagattctggtactggctgagtatcttggtgaggaattcaagaaggctagtgcttga

SEQ ID NO: 3 - protein, 564 aa, Aspergillus niger
MALTSWEQTAAAKRQSVLNAIPEKWRIKGPIPAPSEQRDVTGPYIQQFLSPREVEITETDAVGITERTTTGQWTAVEVTEAFCHRAALAHQLVNCLHEIFFDAALETARILDDHYTKTGKPLGPLHGLPVSLKDQFHVKGVETTMGYVGWINTFQGKTNDPRYLTHESELVKELRAAGAVLYCKTSVPMTLMSGETMNNIITYTHNPKNRLLSSGGSSGGEGALIALRGSPAGFGTDIGGSIRVPASFNGLYGIRPSVGRMPYEGAANSGDGQNTVLSVVGPLSPSARGLILLFKTVLGAMPWLGDPGVLEIPWREEIVEETRKLVQGKPEGLAFGIFYDDGQVKPQPPVERAMRIAAETIKRLGHKLINWEPPSHLTAASLANRAYNMDGGADVLQNFALSNEAIHTSVVIDASGSPQKTALEIAALNVEKREYQKQYLDYWNSTAQLTGTGRPVDAVICPVAPHAACIPGKYATIGYTAFINVLDYTSAVVPVTSADRRVDVVGKEGREYFGELDRKTEGEYDADVFDGAPAGIQLFGRRLQEEKILVLAEYLGEEFKKASA

SEQ ID NO: 4 - DNA, 42 bp, Artificial Sequence
  primer
gtcctgttaattaaccttcaccatggccctcacatcctggga

SEQ ID NO: 5 - DNA, 41 bp, Artificial Sequence
  primer
acttcaggcgcgcctttaagcactagccttcttgaattcct

SEQ ID NO: 6 - DNA, 1954 bp, Aspergillus niger
atgcttcccccctttgattactttacgtatcgtcgcatccggtaggttaccccgtgtgccttcacgcagcaacttaagcagtcctacacagcccacatgcaacttagccgacggctcactgaccattcacagggacctcaagaggagggaacgggcggctcgatttgcatcgctatctcccgattatcatgcgcccttctcggctatcgacagggttatcattaacaagcctatccaggacttggtatatgaggtccagaatgattccttggcacctttagatgtcctacgcacatacggcaaggttgcagtcaaggctcacgaaaagaccaattgcgtgactgagcttctattgcctgaggcagaatcatgggctcagtccgaagtaaacctaaaaggtcccctggcgggtgtgcctatctctttgaaagactcggtgcaagtcaaaggattcgatattactctggggtataccaagttcgcgtgtaaaccgtataaggaggatggtccaatggcaaagctgttgaaggatgctggtaggtgacctgtgccgcgaaacatcacagaaagtatccctgcgctaatgatgaactccatcaggtgcggtcccatatgcgaaaacggcgctgcccgtgacgcttctgtcgttcgaatcagcaaacgctctttggggtcactgccttaacccacatgtcccggaatactctcctggcggctcgacgggtggtgaaggtgctctgctggctcttggtggtcgcatcggtatcgggtcggatgtcgctggctcggttcgcgttcccgctgcctggagcggcatctactccctccgctgtagtactggccgctggcctaaggtcggagtcaacaccagcatggctggccaggaaggtgttgccagtgtcttcagtcctatggcccgtactttgaacgatctcacctatttcaccaaagctattgtcggaatgaagccttggaactacgaccataccgtccaccctatctcctggagagaagatgaggaaattgaagcccaaaacaaaaggttgaggatcggcctcatgagcaacgatggtaagttccaagagcggggacatcaaggacactacttgggcctttctgaccacttgcaggtgtcgttcccccaacgccggccattgaacgtgctatttccaccacagtagccgccctcaccgccgctgggcatactgtatctgagatcacaccccccgctgcggctgacacttttactggtctctccctcgcctcgcagctgctcaactctgatgggtgcgtcacgtttaactcgcatctgcacagctttgagccatccgaccctggtgcagatcagctgacgcggatatgcaatctgccccgtcctctacgttatctctactacctttgggttcggtacatccgacgggatgagaagtgggcgacgctgatccgcgggtttgcccccaagtctgcggcggagctttggaagctcactgcccagcgggaagctttccgggcgacctggcacagctggtgggatgccgagacgcagcagtacgatttcatcctctgccccgtcaatgcgacgccggctctgccccacaaagccatgcacgatgcggtatcctcatgcggatacacgttcctctggaacctgctggactacacagccggtgtcgtgcctgtctcgcacgtggacgcgaagaaggatgctctgtctggtccgtacaagaaggtgctgaaacagctgggagccagcaacgcggtggcctacggtgcctggaagcactacgacgcggcgaagatggcgggattgcctactgcagtgcaggtggtgggacgcagatggcaggaagagaaggtgcttggatacatggcggcagttgaggaggccttggagcagtatcaggacccggtaactggagaaggggggaaatacccattgattgagcttgactag

SEQ ID NO: 7 - DNA, 1740 bp, Aspergillus niger
atgcttcccccctttgattactttacgtatcgtcgcatccgggacctcaagaggagggaacgggcggctcgatttgcatcgctatctcccgattatcatgcgcccttctcggctatcgacagggttatcattaacaagcctatccaggacttggtatatgaggtccagaatgattccttggcacctttagatgtcctacgcacatacggcaaggttgcagtcaaggctcacgaaaagaccaattgcgtgactgagcttctattgcctgaggcagaatcatgggctcagtccgaagtaaacctaaaaggtcccctggcgggtgtgcctatctctttgaaagactcggtgcaagtcaaaggattcgatattactctggggtataccaagttcgcgtgtaaaccgtataaggaggatggtccaatggcaaagctgttgaaggatgctggtgcggtcccatatgcgaaaacggcgctgcccgtgacgcttctgtcgttcgaatcagcaaacgctctttggggtcactgccttaacccacatgtcccggaatactctcctggcggctcgacgggtggtgaaggtgctctgctggctcttggtggtcgcatcggtatcgggtcggatgtcgctggctcggttcgcgttcccgctgcctggagcggcatctactccctccgctgtagtactggccgctggcctaaggtcggagtcaacaccagcatggctggccaggaaggtgttgccagtgtcttcagtcctatggcccgtactttgaacgatctcacctatttcaccaaagctattgtcggaatgaagccttggaactacgaccataccgtccaccctatctcctggagagaagatgaggaaattgaagcccaaaacaaaaggttgaggatcggcctcatgagcaacgatggtgtcgttcccccaacgccggccattgaacgtgctatttccaccacagtagccgccctcaccgccgctgggcatactgtatctgagatcacaccccccgctgcggctgacacttttactggtctctccctcgcctcgcagctgctcaactctgatgggtgcgtcacgtttaactcgcatctgcacagctttgagccatccgaccctggtgcagatcagctgacgcggatatgcaatctgccccgtcctctacgttatctctactacctttgggttcggtacatccgacgggatgagaagtgggcgacgctgatccgcgggtttgcccccaagtctgcggcggagctttggaagctcactgcccagcgggaagctttccgggcgacctggcacagctggtgggatgccgagacgcagcagtacgatttcatcctctgccccgtcaatgcgacgccggctctgccccacaaagccatgcacgatgcggtatcctcatgcggatacacgttcctctggaacctgctggactacacagccggtgtcgtgcctgtctcgcacgtggacgcgaagaaggatgctctgtctggtccgtacaagaaggtgctgaaacagctgggagccagcaacgcggtggcctacggtgcctggaagcactacgacgcggcgaagatggcgggattgcctactgcagtgcaggtggtgggacgcagatggcaggaagagaaggtgcttggatacatggcggcagttgaggaggccttggagcagtatcaggacccggtaactggagaaggggggaaatacccattgattgagcttgactag

SEQ ID NO: 8 - protein, 579 aa, Aspergillus niger
MLPPFDYFTYRRIRDLKRRERAARFASLSPDYHAPFSAIDRVIINKPIQDLVYEVQNDSLAPLDVLRTYGKVAVKAHEKTNCVTELLLPEAESWAQSEVNLKGPLAGVPISLKDSVQVKGFDITLGYTKFACKPYKEDGPMAKLLKDAGAVPYAKTALPVTLLSFESANALWGHCLNPHVPEYSPGGSTGGEGALLALGGRIGIGSDVAGSVRVPAAWSGIYSLRCSTGRWPKVGVNTSMAGQEGVASVFSPMARTLNDLTYFTKAIVGMKPWNYDHTVHPISWREDEEIEAQNKRLRIGLMSNDGVVPPTPAIERAISTTVAALTAAGHTVSEITPPAAADTFTGLSLASQLLNSDGCVTFNSHLHSFEPSDPGADQLTRICNLPRPLRYLYYLWVRYIRRDEKWATLIRGFAPKSAAELWKLTAQREAFRATWHSWWDAETQQYDFILCPVNATPALPHKAMHDAVSSCGYTFLWNLLDYTAGVVPVSHVDAKKDALSGPYKKVLKQLGASNAVAYGAWKHYDAAKMAGLPTAVQVVGRRWQEEKVLGYMAAVEEALEQYQDPVTGEGGKYPLIELD

SEQ ID NO: 9 - DNA, 40 bp, Artificial Sequence
  primer
gtcctgttaattaaccaccatgcttcccccctttgattac

SEQ ID NO: 10 - DNA, 39 bp, Artificial Sequence
  primer
acttcaggcgcgccctagtcaagctcaatcaatgggtat

SEQ ID NO: 11 - DNA, 1735 bp, Aspergillus niger
atgtcgctcctgcgggaagccgagaagtgcttggctaatcagggctctcatgcggctctaaatgcgctaattaccaccctacatcagtcaggacaatggttggaacgtgtgagagatgcggatgcgcggcgcacccggggtgagtatgaaggctgcattgaaaccaatcctcgctttattcagagctgaccaactcgcttcgccaaatagggacgcctaaatctgagatagatggccggttggttgccgtcaaggacaatatgtgcacacgcgaccttccaacaacatgcgcttcaaacgctctggacaaattcgtcagtccgtttaacgcgacagtcgttcaacaattggaggatgcaggcgctgtcgtggccgggaaagctaatctcgacgaatttggaatgggctcccactctattcattcaaactttggccctgtcagaagctcgcgccgtggccaggatgcagaatacctctcagccggtggtagttcgggtggaagtgcggttgctgttgcgacagcccaatgttacgcgtatgtaactctccagattagccttgaatcatacgacgagtaacggtttggcttaacattgctggaacatttacagagcgttgggaactgatacaggcggctccgtgcgattacccgcagcatatacgggaacagtcggcttcaagccttcttatgggctggtctctcgctggggagtggttgcgtacgctaattcactggatacagtcggtgtactgggaagggacgtggccagcgttcgccatgtcttcggtatgtcttgattatgtcggaatttctctcgtctctttgcgtctcgactcactaactcgtgttcgaaacataggcgtcgtgaaccagcatgattcacgcgatcccactaacctttctccctccagtcggtcacggatagactcacatcttaaaatgtctgctttggcatctcggacaagttctcctcttaggataggagtccctctcgagtacaacatatcagagttgtctccatctgcgcgacatgcgtggtctcgctctctggcctatttgcaacgacagggccatagcatccaaacggtgtcccttccaacgaccaaactcgccttatcggcatattacgtactcgcgcctgctgaggcatcctcaaacctggccaagtatgatggcgtcaggtacggaagccgccccgaagcaccagacggaaatgcatcatcagaaaggtatctatatgcgaaaacacgaggtgatgggttcggctccgaagtcaaaagacgaattttgctcggggcatttagcctaagcgctgacgctatggataactacttcattcaagcacagcgggttcgccgccttgtccaacatgacttcaacgccgcgtttagggcatgccagccattgatttcctcgcaacttggatcaaagtcgggttctgcagacaccggtgtagatgttctcatttgcccgacagcgccatcatcaccaccacgcatttcaagccttataggacctgatgcgacaacttcgcctttggaggcatatacgaatgatgtatttactgtgcctgctagtttggcaggtcttccggctatatctgtgccggtcactacagaaaatgctgatggcgccgaggatggagatctggctggaatccaagtaattggccaatacggtgatgatgagcttgtgttaaaggttgccgagctgctcgagggcaggcacctatga

SEQ ID NO: 12 - DNA, 1515 bp, Aspergillus niger
atgtcgctcctgcgggaagccgagaagtgcttggctaatcagggctctcatgcggctctaaatgcgctaattaccaccctacatcagtcaggacaatggttggaacgtgtgagagatgcggatgcgcggcgcacccgggggacgcctaaatctgagatagatggccggttggttgccgtcaaggacaatatgtgcacacgcgaccttccaacaacatgcgcttcaaacgctctggacaaattcgtcagtccgtttaacgcgacagtcgttcaacaattggaggatgcaggcgctgtcgtggccgggaaagctaatctcgacgaatttggaatgggctcccactctattcattcaaactttggccctgtcagaagctcgcgccgtggccaggatgcagaatacctctcagccggtggtagttcgggtggaagtgcggttgctgttgcgacagcccaatgttacgcagcgttgggaactgatacaggcggctccgtgcgattacccgcagcatatacgggaacagtcggcttcaagccttcttatgggctggtctctcgctggggagtggttgcgtacgctaattcactggatacagtcggtgtactgggaagggacgtggccagcgttcgccatgtcttcggcgtcgtgaaccagcatgattcacgcgatcccactaacctttctccctccagtcggtcacggatagactcacatcttaaaatgtctgctttggcatctcggacaagttctcctcttaggataggagtccctctcgagtacaacatatcagagttgtctccatctgcgcgacatgcgtggtctcgctctctggcctatttgcaacgacagggccatagcatccaaacggtgtcccttccaacgaccaaactcgccttatcggcatattacgtactcgcgcctgctgaggcatcctcaaacctggccaagtatgatggcgtcaggtacggaagccgccccgaagcaccagacggaaatgcatcatcagaaaggtatctatatgcgaaaacacgaggtgatgggttcggctccgaagtcaaaagacgaattttgctcggggcatttagcctaagcgctgacgctatggataactacttcattcaagcacagcgggttcgccgccttgtccaacatgacttcaacgccgcgtttagggcatgccagccattgatttcctcgcaacttggatcaaagtcgggttctgcagacaccggtgtagatgttctcatttgcccgacagcgccatcatcaccaccacgcatttcaagccttataggacctgatgcgacaacttcgcctttggaggcatatacgaatgatgtatttactgtgcctgctagtttggcaggtcttccggctatatctgtgccggtcactacagaaaatgctgatggcgccgaggatggagatctggctggaatccaagtaattggccaatacggtgatgatgagcttgtgttaaaggttgccgagctgctcgagggcaggcacctatga

SEQ ID NO: 13 - protein, 504 aa, Aspergillus niger
MSLLREAEKCLANQGSHAALNALITTLHQSGQWLERVRDADARRTRGTPKSEIDGRLVAVKDNMCTRDLPTTCASNALDKFVSPFNATVVQQLEDAGAVVAGKANLDEFGMGSHSIHSNFGPVRSSRRGQDAEYLSAGGSSGGSAVAVATAQCYAALGTDTGGSVRLPAAYTGTVGFKPSYGLVSRWGVVAYANSLDTVGVLGRDVASVRHVFGVVNQHDSRDPTNLSPSSRSRIDSHLKMSALASRTSSPLRIGVPLEYNISELSPSARHAWSRSLAYLQRQGHSIQTVSLPTTKLALSAYYVLAPAEASSNLAKYDGVRYGSRPEAPDGNASSERYLYAKTRGDGFGSEVKRRILLGAFSLSADAMDNYFIQAQRVRRLVQHDFNAAFRACQPLISSQLGSKSGSADTGVDVLICPTAPSSPPRISSLIGPDATTSPLEAYTNDVFTVPASLAGLPAISVPVTTENADGAEDGDLAGIQVIGQYGDDELVLKVAELLEGRHL

SEQ ID NO: 14 - DNA, 1760 bp, Aspergillus niger
atggcgaacacaacgtgggagcatagagccaagagcaagcaagctcagacggcagcagcgattccaccagaatggactttaccagccgatatacttctcgactcctcggcgaatgtcctcgatgtgcccaggacctgtgggctcctgaccgaaagagaaatccatattaccgaagactatgatgccacagcattacttgaaaagttggctaccggagagttcagctcactcgaggtcacgacagcattctgcaagcgggctgcaattgcgcagcagttgacctgctgcttgacggaaatcttcttcgacaaagcactagccagagcaaagcagctcgacgaaattctggctcaaactggggtaacaaccgggccactgcatggcctgcccataagtatcaaggagtcgttcaatgtccctggagttccgactacactggggttcgtcggatttctcgatcgggccccggcgtccatgagttccgccctggttgagatcctgaacaactgtggggctgttctatatgtcaagaccaatgtcccccagacaatgatgactccggactcccataacaatgtcttcggtcgtgtgctcaacccgcacggacggagcctgaccgctggaggcagcagtggcggagagggagcattggtggcgatgaggggctcagtcctcggcgtggggacagatatcgccggctccattcgcatccccgccctctgctgtggtgtttttggtttcaaacccacggcttgccgtatcccatatgcaggtcaaacgtctgccgggcgtccgggtatgacgggcatcctgccgtccgccgggcccatgtgccattctatccgcgatgcagaactctttctcaaagttgtcctcaactcgaggcctgccgatctggatgactatgcactcgacattccatggtcccctgctccgcaaaaggagaggcttactattgggctcctgcccgaggacccgtcgttcccgcttcatccaccgatgcgacgtaccctgaacgccgcagtgaaggcgctgactacagccggacaccgggtcatcgacctctcaggacaggctccgtccttctccaatgcatgttctctggcattgcgatatttcggcctggacccggatcgaactgcactgcgccacataacgcaggcgggcgaacctttcattccttcgctcaaattcacctatgatctcaatgagccgacacgtgagcccaccttgagagatctattcgaatcgaatgtcgcccgtagtcagctcgcaacccaggcacgaaagctcttcgttgataatcagcttgatctgttcctgggcgcggcttatcaaagcacatcggttccccatgatacctacggtattccggtatataccgtgctttggaacctgattaatgtgggtatcccacactgcctttcttgttttctttttcttttttgtttcccccccttctttctgctccaattgcatggggtgtctgctaatgatgcactagtatccggcgtgcgttatcccatttgggcaggccaacaaggtagaggacgcagcgtataaccgcgacgtctcttatgtgccagactgtaagttgcgcccctcaattggtctcgctcagtctactaactactgtagatcggccagaagaaattgaaggcgcaccgtgtcacatccatctgataggcagaccgatggcggacgaaaaattggttcaggatgcgaagactatatgcaccgtaatgagtgatgtctcttga

SEQ ID NO: 15 - DNA, 1611 bp, Aspergillus niger
atggcgaacacaacgtgggagcatagagccaagagcaagcaagctcagacggcagcagcgattccaccagaatggactttaccagccgatatacttctcgactcctcggcgaatgtcctcgatgtgcccaggacctgtgggctcctgaccgaaagagaaatccatattaccgaagactatgatgccacagcattacttgaaaagttggctaccggagagttcagctcactcgaggtcacgacagcattctgcaagcgggctgcaattgcgcagcagttgacctgctgcttgacggaaatcttcttcgacaaagcactagccagagcaaagcagctcgacgaaattctggctcaaactggggtaacaaccgggccactgcatggcctgcccataagtatcaaggagtcgttcaatgtccctggagttccgactacactggggttcgtcggatttctcgatcgggccccggcgtccatgagttccgccctggttgagatcctgaacaactgtggggctgttctatatgtcaagaccaatgtcccccagacaatgatgactccggactcccataacaatgtcttcggtcgtgtgctcaacccgcacggacggagcctgaccgctggaggcagcagtggcggagagggagcattggtggcgatgaggggctcagtcctcggcgtggggacagatatcgccggctccattcgcatccccgccctctgctgtggtgtttttggtttcaaacccacggcttgccgtatcccatatgcaggtcaaacgtctgccgggcgtccgggtatgacgggcatcctgccgtccgccgggcccatgtgccattctatccgcgatgcagaactctttctcaaagttgtcctcaactcgaggcctgccgatctggatgactatgcactcgacattccatggtcccctgctccgcaaaaggagaggcttactattgggctcctgcccgaggacccgtcgttcccgcttcatccaccgatgcgacgtaccctgaacgccgcagtgaaggcgctgactacagccggacaccgggtcatcgacctctcaggacaggctccgtccttctccaatgcatgttctctggcattgcgatatttcggcctggacccggatcgaactgcactgcgccacataacgcaggcgggcgaacctttcattccttcgctcaaattcacctatgatctcaatgagccgacacgtgagcccaccttgagagatctattcgaatcgaatgtcgcccgtagtcagctcgcaacccaggcacgaaagctcttcgttgataatcagcttgatctgttcctgggcgcggcttatcaaagcacatcggttccccatgatacctacggtattccggtatataccgtgctttggaacctgattaattatccggcgtgcgttatcccatttgggcaggccaacaaggtagaggacgcagcgtataaccgcgacgtctcttatgtgccagactatcggccagaagaaattgaaggcgcaccgtgtcacatccatctgataggcagaccgatggcggacgaaaaattggttcaggatgcgaagactatatgcaccgtaatgagtgatgtctcttga

SEQ ID NO: 16 - protein, 536 aa, Aspergillus niger
MANTTWEHRAKSKQAQTAAAIPPEWTLPADILLDSSANVLDVPRTCGLLTEREIHITEDYDATALLEKLATGEFSSLEVTTAFCKRAAIAQQLTCCLTEIFFDKALARAKQLDEILAQTGVTTGPLHGLPISIKESFNVPGVPTTLGFVGFLDRAPASMSSALVEILNNCGAVLYVKTNVPQTMMTPDSHNNVFGRVLNPHGRSLTAGGSSGGEGALVAMRGSVLGVGTDIAGSIRIPALCCGVFGFKPTACRIPYAGQTSAGRPGMTGILPSAGPMCHSIRDAELFLKVVLNSRPADLDDYALDIPWSPAPQKERLTIGLLPEDPSFPLHPPMRRTLNAAVKALTTAGHRVIDLSGQAPSFSNACSLALRYFGLDPDRTALRHITQAGEPFIPSLKFTYDLNEPTREPTLRDLFESNVARSQLATQARKLFVDNQLDLFLGAAYQSTSVPHDTYGIPVYTVLWNLINYPACVIPFGQANKVEDAAYNRDVSYVPDYRPEEIEGAPCHIHLIGRPMADEKLVQDAKTICTVMSDVS

SEQ ID NO: 17 - DNA, 1990 bp, Aspergillus niger
atgactatcccaacctggaaatcaaccgtgcacaagaagcgcgcagctcagctggccgccattccacctgattggcgtctcccggagtccatcactgccgatccgccactcaatgctcttgaggcaatccgctcttgtgatattctcacggagaaagagcaggaatggacggagattaatgatagtactgtgctgctttccatgctggcgaaccgtgagattagctccgttcagcttacgacggcgttctgtaagcgcgcggccgtcgcccagcagcttaccaagtgcttgacggagattttcttcgatagggcgctagcacgggctaaggagctcgatgatattttggaaaagacaggaaaggtgacggggccgttgcatgggctgccggtttcaattaaggatcggtttgatgtcgagggatatgatacaaccgttggttcgtatccatgttctagcatcactgctgctgggagaggagccttggctaactagtggtatgtaggctgggttgggctagtgggcaaacccgcgcaaagctccagcagtattgccttgtcgttggagtcaatgggcgcagtattgtatgtcaagacgaatgtaccacagtctttgatggtatgcaatcacccttttacatttcgcatgatgggagattctgattaagtcagatgtctgattcctacaaccatgtgttcggacaatccatcaatgccttcaacaaacaactcatctccggtgggagttccggcggcgaaggcgccctcattggcacgggtggcagtgtcgttggtatagggacagatatcgggggctcgattcgtatcccggccaatttgcagggtttatactcaatctgtcctactacaggtcgagtgccatggaactgctcgttgtacgtgtccatctctatctactgattctagaccatagctgaccatacccagcatgcaccagcactaccttgttcctccagtcgcaggacccatggcccgcagtctttccagcattgaacatttcatgcaatccctcctcgactccaatccttggaatattgatccgggctgcatcccaattccctggagaaaggaacttgcagcgaagcccactggtaaactacgactcggaattgtctacgacgacggcgtggtaaaaccacagcccccaatcgccagggcaatgcgcgaagttgcaaagaaactcaaagaagctggccatgaaggtgtgtacacaatctacaatctccctcttacctcatcaatcagctttcactgactagacaagtcttcgaatgggacacctccctccacaccacaggccaaaacctctggacaaaagccatcctcgccgacggaggccaacactgtcgctatctatgcgacattgttggagagccactgatagaagggatggttgtagggacagaaaaagatgagctgaccatctcagaacgcgaagaggtcggtcccataccctaatcttctacctctccaatgcttatattaatacaaacatatagctcgaagagaccaaatgggcctaccaaaccagcttcctccagcaatggacatcaagcaacatcgacgccctcctcatgcccgtcacgccctgggtcggctaccggccaaaatcctgggtcgtcagctcccagtggctcggctacacggctctctggaacttgctcaactacgcggctgttactgttccggtggctaaagtggatggagcacttgatcagccggatcaggagtggttggagcatgtgcctaggaatgactcggatcggtttaattgggaacagtgtatgcatactctcctttccatacatacggttatcggggataatagctaaccctatcactatgatctagatgatcccgacctggtcaaagacatgcccatcactgtacaaatcgtcggaggccgctttggcgaagaaaaggccgtttcggtagccaaggtagtagatgaggttttgcaggagccctag

SEQ ID NO: 18 - DNA, 1494 bp, Aspergillus niger
atgactatcccaacctggaaatcaaccgtgcacaagaagcgcgcagctcagctggccgccattccacctgattggcgtctcccggagtccatcactgccgatccgccactcaatgctcttgaggcaatccgctcttgtgatattctcacggagaaagagcaggaatggacggagattaatgatagtactgtgctgctttccatgctggcgaaccgtgagattagctccgttcagcttacgacggcgttctgtaagcgcgcggccgtcgcccagcagcttaccaagtgcttgacggagattttcttcgatagggcgctagcacgggctaaggagctcgatgatattttggaaaagacaggaaaggtgacggggccgttgcatgggctgccggtttcaattaaggatcggtttgatgtcgagggatatgatacaaccgttggctgggttgggctagtgggcaaacccgcgcaaagctccagcagtattgccttgtcgttggagtcaatgggcgcagtattgtatgtcaagacgaatgtaccacagtctttgatgatgtctgattcctacaaccatgtgttcggacaatccatcaatgccttcaacaaacaactcatctccggtgggagttccggcggcgaaggcgccctcattggcacgggtggcagtgtcgttggtatagggacagatatcgggggctcgattcgtatcccggccaatttgcagggtttatactcaatctgtcctactacaggtcgagtgccatggaactgctcgttcatgcaccagcactaccttgttcctccagtcgcaggacccatggcccgcagtctttccagcattgaacatttcatgcaatccctcctcgactccaatccttggaatattgatccgggctgcatcccaattccctggagaaaggaacttgcagcgaagcccactggtaaactacgactcggaattgtctacgacgacggcgtggtaaaaccacagcccccaatcgccagggcaatgcgcgaagttgcaaagaaactcaaagaagctggccatgaagggacagaaaaagatgagctgaccatctcagaacgcgaagagctcgaagagaccaaatgggcctaccaaaccagcttcctccagcaatggacatcaagcaacatcgacgccctcctcatgcccgtcacgccctgggtcggctaccggccaaaatcctgggtcgtcagctcccagtggctcggctacacggctctctggaacttgctcaactacgcggctgttactgttccggtggctaaagtggatggagcacttgatcagccggatcaggagtggttggagcatgtgcctaggaatgactcggatcggtttaattgggaacagtatgatcccgacctggtcaaagacatgcccatcactgtacaaatcgtcggaggccgctttggcgaagaaaaggccgtttcggtagccaaggtagtagatgaggttttgcaggagccctag

SEQ ID NO: 19 - protein, 497 aa, Aspergillus niger
MTIPTWKSTVHKKRAAQLAAIPPDWRLPESITADPPLNALEAIRSCDILTEKEQEWTEINDSTVLLSMLANREISSVQLTTAFCKRAAVAQQLTKCLTEIFFDRALARAKELDDILEKTGKVTGPLHGLPVSIKDRFDVEGYDTTVGWVGLVGKPAQSSSSIALSLESMGAVLYVKTNVPQSLMMSDSYNHVFGQSINAFNKQLISGGSSGGEGALIGTGGSVVGIGTDIGGSIRIPANLQGLYSICPTTGRVPWNCSFMHQHYLVPPVAGPMARSLSSIEHFMQSLLDSNPWNIDPGCIPIPWRKELAAKPTGKLRLGIVYDDGVVKPQPPIARAMREVAKKLKEAGHEGTEKDELTISEREELEETKWAYQTSFLQQWTSSNIDALLMPVTPWVGYRPKSWVVSSQWLGYTALWNLLNYAAVTVPVAKVDGALDQPDQEWLEHVPRNDSDRFNWEQYDPDLVKDMPITVQIVGGRFGEEKAVSVAKVVDEVLQEP

SEQ ID NO: 20 - DNA, 2017 bp, Artificial Sequence
  synthetic DNA amplified by primers of SEQ ID NOS 4 and 5
gtcctgttaattaaccttcaccatggccctcacatcctgggaacaaaccgcagcggccaaacgccaatccgtcctcaacgccatccccgagaaatggcgcatcaagggtcctatccccgcaccgtcggagcagcgcgacgtaacaggcccctacatccagcagttcctatccccacgcgaggttgaaatcaccgaaacagacgccgtagggatcacagagcgaactacaacgggccagtggacagctgtggaggtgaccgaggcgttctgccatcgcgcagcattggcgcatcaactcgtacattccccatccacaaggagtgctagtctgcgctttactaatcgagaaaaaggtaaactgcttgcatgaaatcttcttcgatgccgcgcttgaaaccgcccgcattctagacgaccactacaccaagaccggcaagccactcggtccccttcacggcctccctgtcagtctgaaggatcaattccacgtcaagggcgtagaaacaaccatgggttacgtcggctggataaacaccttccaaggcaagaccaatgacccgcgctatcttacacacgaaagcgaactcgttaaagaactccgcgccgcgggagccgtcctctactgcaagactagcgtccccatgacgttgatgtcaggtgaaaccatgaacaatatcataacttacacacataacccgaagaacaggcttctcagttctggaggtagttccgggggcgaaggagcactgatcgcgttgcggggatcaccagccgggtttggtacggatatcgggggtagtatccgtgttcctgcgtcgttcaatggactgtatgggatacggccgtctgtggggagaatgccgtacgagggggcggccaattcgggcgatggacagaatactgtgttgtcggttgtggggccgttgtctccttcggcgagagggttgatattgctgttcaagacggtgttgggggcaatgccgtggttgggagatcctggtgtgttggagattccctggagggaggaaatcgtagaggagacgagaaaattagtgcagggaaagccagaggggctagcttttggaatattctacgatgatggtcaggtaaagccgcagccaccggtcgagagagcgatgcggattgctgcagagacgatcaagcgtctaggacataaggtgagtgccctccttcttcttgcgacactgctaacattcatcccagctcatcaattgggaacccccctctcacctaacagccgcctccctcgcagtaagtcccccatccaacccactacaccacaaccccctaacaataaaccaacccccagaaccgcgcctacaacatggacggcggcgccgacgtactccaaaacttcgccctgtccaacgaagccatccacacctccgtagtaatcgacgcatcaggatccccccaaaagaccgcactagagatcgccgcgctaaacgtcgagaagcgcgaataccagaaacaataccttgactactggaacagcacggcgcaattgacagggactggacgacccgtcgacgcggtcatttgtccagtggcgccgcatgcggcgtgcattccggggaagtatgcgacgatcgggtatacggcgtttattaatgtgttggattatacgagtgcggttgtgccggttacgagtgctgataggagggtggatgttgtagggaaggaaggaagggagtattttggggagttggataggaagaccgagggggagtgtaagttcttccctttcttttcttctttcttttcattgagctatccaatttggttggaggtcttgtgtgtttgtttgttcggagagtggtgatggggttatgtgctgactggatgtttctatctagacgatgcggatgtgtttgatggggcgccggctgggattcagctctttggaagacggcttcaggaggagaagattctggtactggctgagtatcttggtgaggaattcaagaaggctagtgcttaaaggcgcgcctgaagt

SEQ ID NO: 21 - DNA, 1987 bp, Artificial Sequence
  synthetic DNA amplified by primers of SEQ ID NOS 9 and 10
gtcctgttaattaaccaccatgcttcccccctttgattactttacgtatcgtcgcatccggtaggttaccccgtgtgccttcacgcagcaacttaagcagtcctacacagcccacatgcaacttagccgacggctcactgaccattcacagggacctcaagaggagggaacgggcggctcgatttgcatcgctatctcccgattatcatgcgcccttctcggctatcgacagggttatcattaacaagcctatccaggacttggtatatgaggtccagaatgattccttggcacctttagatgtcctacgcacatacggcaaggttgcagtcaaggctcacgaaaagaccaattgcgtgactgagcttctattgcctgaggcagaatcatgggctcagtccgaagtaaacctaaaaggtcccctggcgggtgtgcctatctctttgaaagactcggtgcaagtcaaaggattcgatattactctggggtataccaagttcgcgtgtaaaccgtataaggaggatggtccaatggcaaagctgttgaaggatgctggtaggtgacctgtgccgcgaaacatcacagaaagtatccctgcgctaatgatgaactccatcaggtgcggtcccatatgcgaaaacggcgctgcccgtgacgcttctgtcgttcgaatcagcaaacgctctttggggtcactgccttaacccacatgtcccggaatactctcctggcggctcgacgggtggtgaaggtgctctgctggctcttggtggtcgcatcggtatcgggtcggatgtcgctggctcggttcgcgttcccgctgcctggagcggcatctactccctccgctgtagtactggccgctggcctaaggtcggagtcaacaccagcatggctggccaggaaggtgttgccagtgtcttcagtcctatggcccgtactttgaacgatctcacctatttcaccaaagctattgtcggaatgaagccttggaactacgaccataccgtccaccctatctcctggagagaagatgaggaaattgaagcccaaaacaaaaggttgaggatcggcctcatgagcaacgatggtaagttccaagagcggggacatcaaggacactacttgggcctttctgaccacttgcaggtgtcgttcccccaacgccggccattgaacgtgctatttccaccacagtagccgccctcaccgccgctgggcatactgtatctgagatcacaccccccgctgcggctgacacttttactggtctctccctcgcctcgcagctgctcaactctgatgggtgcgtcacgtttaactcgcatctgcacagctttgagccatccgaccctggtgcagatcagctgacgcggatatgcaatctgccccgtcctctacgttatctctactacctttgggttcggtacatccgacgggatgagaagtgggcgacgctgatccgcgggtttgcccccaagtctgcggcggagctttggaagctcactgcccagcgggaagctttccgggcgacctggcacagctggtgggatgccgagacgcagcagtacgatttcatcctctgccccgtcaatgcgacgccggctctgccccacaaagccatgcacgatgcggtatcctcatgcggatacacgttcctctggaacctgctggactacacagccggtgtcgtgcctgtctcgcacgtggacgcgaagaaggatgctctgtctggtccgtacaagaaggtgctgaaacagctgggagccagcaacgcggtggcctacggtgcctggaagcactacgacgcggcgaagatggcgggattgcctactgcagtgcaggtggtgggacgcagatggcaggaagagaaggtgcttggatacatggcggcagttgaggaggccttggagcagtatcaggacccggtaactggagaaggggggaaatacccattgattgagcttgactagggcgcgcctgaagt

(19) United States
(12) Patent Application Publication
(10) Pub. No.: US 2008/0070277 A1
(43) Pub. Date: Mar. 20, 2008

(54) Homologous Amds Genes as Selectable Marker

(76) Inventors: Cornelis Maria Jacobus Sagt, Utrecht (NL); Johannes Hendrik de Winde, Voorhout (NL); Thibaut Jose Wenzel, Leiden (NL); Brenda Vonk, Spijkenisse (NL)

Correspondence Address: NIXON & VANDERHYE, PC, 901 NORTH GLEBE ROAD, 11TH FLOOR, ARLINGTON, VA, 22203, US

(21) Appl. No.: 11/664,585

(22) PCT Filed: Oct. 17, 2005
(86) PCT No.: PCT/EP05/55316

§ 371 Date: May 10, 2007

(30) Foreign Application Priority Data
Oct. 15, 2004 (EP) 04105077.4
Dec. 29, 2004 (EP) 04107053.3

(51) Int. Cl. C12P 21/04; C12N 1/00; C12N 15/00; C12N 15/11; C12N 9/00

(52) U.S. Cl. 435/69.1; 435/183; 435/254.11; 435/254.3; 435/254.5; 435/254.6; 435/320.1; 536/23.2

(57) ABSTRACT

The present invention relates to novel functional amdS genes from A. niger that can be used as dominant and bi-directional selection marker gene in the transformation of organisms. The present invention further relates to the production of a compound of interest in a fungal host cell transformed with the amdS genes of the invention. Preferred fungal host cells are filamentous fungal cells. The amdS genes of the invention provide means for identification of functional homologues in other Aspergillus species.

### FIELD OF THE INVENTION

The present invention relates to the field of molecular biology, in particular the invention is concerned with selectable marker genes to be used in transformation of organisms.

### BACKGROUND OF THE INVENTION

The Aspergillus nidulans amdS gene is probably the most frequently used selectable marker for the transformation of filamentous fungi and has been applied in most of the industrially important filamentous fungi such as e.g. Aspergillus niger (Kelly and Hynes 1985, EMBO J. 4: 475-479), Penicillium chrysogenum (Beri and Turner 1987, Curr. Genet. 11: 639-641), Trichoderma reesei (Pentillä et al. 1987, Gene 61: 155-164), Aspergillus oryzae (Christensen et al. 1988, Bio/technology 6: 1419-1422) and Trichoderma harzianum (Pe'er et al. 1991, Soil Biol. Biochem. 23: 1043-1046).

The popularity of the amdS gene as a selectable marker is most likely a result of the fact that it is the only available non-antibiotic marker gene, which can be used as a dominant selectable marker in the transformation of fungi. Dominant selectable markers provide the advantage that they can be used directly in any strain without the requirement for mutant recipient strains. The antibiotic-resistance genes are, however, not preferred for use in industrial strains because the regulatory authorities in most countries object to the use of antibiotic markers in view of the potential risks of spread of antibiotic-resistance genes in the biosphere upon large-scale use of production strains carrying such genes.

The amdS gene has been used as a dominant marker even in fungi known to contain an endogenous amdS gene, i.e. A. nidulans (Tilburn et al. 1983, Gene 26: 205-221) and A. oryzae (Gomi et al. 1991, Gene 108: 91-98). In these cases the background of non-transformants can be suppressed by the inclusion of CsCl in the selection medium. In addition, high-copynumber transformants are provided with a growth advantage over the non-transformants (when acetamide is the sole nitrogen-source) because of the higher gene dosage.

In addition to its dominant character, the amdS selectable marker provides the advantage of being a bidirectional marker. This means that, apart from the positive selection for the presence of the amdS gene using acetamide as sole carbon- or nitrogen-source, a counterselection can be applied using fluoracetamide to select against the presence of the amdS gene (Hynes and Pateman 1970, Mol. Gen. Genet. 108, 107-106). The fluoracetamide counterselection has been applied to cure genetically engineered strains from recombinant constructs carrying the amdS gene (e.g. Ward et al. 1993, Appl. Microbiol. Biotechnol. 39, 738-743).

A disadvantage of the amdS marker is the fact that the A. nidulans amdS gene is a heterologous gene in industrial fungi such as A. niger, A. oryzae, T. reesei and P. chrysogenum. Even though this may seem trivial to most molecular biologists, regulatory authorities often object that production strains containing the heterologous A. nidulans amdS gene posses a new (the gene being heterologous) and unnecessary (the marker gene not being necessary once the transformant strain is obtained) property, the risks of which cannot be foreseen.

We have previously addressed this problem by developing a method to obtain recombinant fungal production strains that are free of selectable markers (EP-A-0 635 574). In this method, the bidirectionality of the amdS marker is used to remove the marker from specially constructed expression cassettes once they have been introduced in the fungal genome. The method is, however, less compatible with the high copy numbers which are often necessary in industrial production strains. For these situations, a homologous and dominant selectable marker would still be required.

Since the first report on the use of A. nidulans amdS gene as a homologous dominant marker, considerate research efforts led to the discovery of several other amdS genes to be used as homologous selection marker e.g. in A. oryzae, S. cerevisiae, P. chrysogenum (described in EP0758020A2). However, in the scientific world it has been doubted that A. niger contains any functional amdS gene at all that could be used as dominant markers (Debets et al., Mol. Gen. Genet. (1990) 222: 284-290; Debets et al., Mol. Gen. Genet. (1990) 224: 264-268; Finkelstein and Ball, Biotechnology of Filamentous fungi; Technology and products (1992) ISBN 0-7506-9115-8). This prejudice has vastly strengthened over the years, since no amdS gene was identified in A. niger for almost a decade. Last, a functional amdS gene was identified in A. niger (described in EP0758020A2).

However, there is still a need for dominant and bi-directional selection marker genes.

### DESCRIPTION OF THE FIGURES

FIG. 1 depicts the A. niger expression vector pGBFIN-32.

FIG. 2 depicts the A. niger expression vector pGBFINAMD-2, for expression of the novel acetamidse-encoding gene AMD2.

FIG. 3 depicts the A. niger expression vector pGBFINAAE-1, for expression of the novel acetamidase-encoding gene AEE1.

### DETAILED DESCRIPTION OF THE INVENTION

Several terms used in the present description and claims are defined as follows.

The term “gene” is herein defined as a DNA sequence encoding a polypeptide, irrespective of whether the DNA sequence is a cDNA or a genomic DNA sequence, which may contain one or more introns.

The term “selection marker gene” (or selectable marker gene) is herein defined as a gene that encodes a polypeptide that provides a phenotype to the cell containing the gene such that the phenotype allows either positive or negative, selection of cells containing the selection marker gene. The selection marker gene may be used to distinguish between transformed and non-transformed cells or may be used to identify cells having undergone recombination or other kinds of genetic modifications.

An “acetamidase” is herein defined as an enzyme which is capable of catalysing the hydrolysis of acetamide into acetic acid and ammonium, and/or which is capable of catalysing the hydrolysis of related amide-compounds such as acrylamide or ù-amino acids.

An “amdS gene” is herein defined as a gene, which is preferably obtainable from a filamentous fungus, and which encodes a polypeptide that is an acetamidase as defined above. Preferably an amdS gene shows sequence similarity with one or more of the amdS genes known in the art, i.e. the amdS genes from A. nidulans, A. oryzae, A. niger, P. chrysogenum or the amdS-like gene from S. cerevisiae. An amdS gene preferably encodes a protein of about 500 to 600 amino acids. An amdS gene is therefore usually contained within a DNA fragment of about 2.0 kb. Of course the presence of introns in a genomic amdS gene can increase the length to e.g. about 2.5 kb or more.

The terms “homologous” gene is herein defined as a gene that is obtainable from a strain that belongs to the same species, including variants thereof, as does the strain actually containing the gene. Preferably, the donor and acceptor strain are the same. It is to be understood that the same applies to polypeptides encoded by homologous genes. Fragments and mutants of genes are also considered homologous when the gene from which the mutants or fragments are derived is a homologous gene. Also non-native combinations of regulatory sequences and coding sequences are considered homologous as long as the coding sequence is homologous. It follows that the term heterologous herein refers to genes or polypeptides for which donor and acceptor strains do not belong to the same species or variants thereof.

The term “endogenous” gene is herein defined as a naturally occurring copy of a gene in the genome of the organism in question.

The term “fungus” herein refers to all members of the division Eumycota of the kingdom Fungi and thus includes all filamentous fungi and yeasts.

“Filamentous fungi” include all filamentous forms of the subdivision Eumycota and Oomycota (as defined by Hawksworth et al., 1995, supra). The filamentous fungi are characterized by a mycelia wall composed of chitin, cellulose, glucan, chitosan, mannan, and other complex polysaccharides. Vegetative growth is by hyphal elongation and carbon catabolism is obligately aerobic. Filamentous fungal strains include, but are not limited to, strains of Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, and Trichoderma.

In view of the nomenclature of black Aspergilli, the term Aspergillus niger is herein defined as including all (black) Aspergilli that can be found in the Aspergillus niger Group as defined by Raper and Fennell (1965, In: The Genus Aspergillus, The Williams & Wilkins Company, Baltimore, pp 293-344). Similarly, also for the other Aspergillus species we will refer to the Aspergillus groups as defined by Raper and Fennell supra, thereby including all species and variants included in a particular group by these authors.

Since the first report on the use of A. nidulans amdS gene as a homologous dominant marker, considerate research efforts led to the discovery of several other amdS genes to be used as homologous selection markers e.g. in A. oryzae, S. cerevisiae, P. chrysogenum (described in EP0758020A2) and in A. niger (described in EP0758020A2).

Surprisingly, we discovered five novel putative amdS genes in A. niger. The novel amdS genes of the invention can be used as homologous selectable marker genes, which is herein understood to mean that the amdS genes are used to select transformants of the same species as the species from which the amdS gene was originally derived. This offers the advantage that the transformants obtained do not contain a foreign selectable marker gene. In principle this allows to construct recombinant strains which contain no foreign DNA other than absolutely necessary, i.e. the (heterologous) gene of interest to be expressed.

In a first aspect, the invention relates to a DNA sequence derivable from an Aspergillus, preferably an Aspergillus niger and encoding an acetamidase, wherein the DNA sequence is not SEQ ID NO: 18 described in EP0758020A2, but is selected from the group consisting of:
a. a DNA sequence having the nucleotide sequence of SEQ ID NO: 1, SEQ ID NO: 6, SEQ ID NO: 11, SEQ ID NO: 14, or SEQ ID NO: 17, and
b. fragments or mutants of any one of the DNA sequences of (a).

In a preferred embodiment, the acetamidase encoded by the sequence according to (a) or (b) comprises an amino acid sequence, wherein the amino acid positional identity with one of the sequences of SEQ ID NO: 3, SEQ ID NO: 8, SEQ ID NO: 13, SEQ ID NO: 16, or SEQ ID NO: 19 is more than 40%. Preferably, the match percentage, i.e. positional identity is at least about 50%, more preferably at least about 60%, even more preferably at least about 70%, even more preferably at least about 80%, even more preferably at least about 85%, even more preferably at least about 90%, even more preferably at least about 95%, even more preferably at least about 97%, even more preferably at least about 98%, even more preferably at least about 99% identity, and most preferably, the match percentage i.e. identity is equal to 100%.

For purposes of the present invention, the degree of identity, i.e. the match percentage, between two polypeptides, respectively two nucleic acid sequences is preferably determined using the optimal global alignment method CDA (Huang, 1994, A Context Dependent Method for Comparing Sequences, Proceedings of the 5th Symposium on Combinatorial Pattern Matching, Lecture Notes in Computer Science 807, Springer-Verlag, 54-63) with the parameters set as follows: (i) for (poly)peptide alignments: Mismatch:−2 GapOpen:11 GapExtend:1 ContextLength:10 MatchBonus:1, and (ii) for nucleotide sequence alignments Mismatch:−15 GapOpen:5 GapExtend:2 ContextLength:10 MatchBonus:1.

The terms “degree of identity”, “identity” and “match percentage” are used interchangeably to indicate the degree of identity between two polypeptides or nucleic acid sequences as calculated by the optimal global alignment method indicated above.

Examples of alternative programs used for alignments and determination of homology are Clustal method (Higgins, 1989, CABIOS 5: 151-153), the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.), BLAST (NCBI), GAP (Huang) for the optimal global alignments, MAP (Huang), MultiBLAST (NCBI), ClustalW, Cap Assembler and Smith Waterman for multiple alignments.

### REFERENCES

Pairwise alignment: (1) BLAST, (2) GAP, (3) MAP,
(4) Smith Waterman, and (5) Cap Assembler

| (1) Tatusova T A and | BLAST 2 sequences, a new tool for |
|---|---|
| Madden T L (1999) | comparing protein and nucleotide |
| | sequences. FEMS Microbiol Lett 174: 247- |
| | 50 |
| (2) (3) Huang X (1994) | On global sequence alignment. Comput |
| | Appl Biosci 10: 227-35 |
| (4) Smith T F and | Identification of common molecular |
| Waterman M S (1981) | subsequences. J Mol Biol 147: 195-197 |
| (5) Huang X (1992) | A contig assembly program based on |
| | sensitive detection of fragment overlaps |
| | Genomics 14: 18-25 |
| (5) Huang X (1996) | An improved sequence assembly program. |
| | Genomics 33: 21-31 |
| (6) Thompson J D, | CLUSTAL W: improving the sensitivity of |
| Higgins D G, and | progressive multiple sequence alignment |
| Gibson T J (1994) | through sequence weighting, positions- |
| | specific gap penalties and weight matrix |
| | choice. Nucleic Acids Research 22: 4673- |
| | 4680 |

The techniques used to isolate or clone a nucleic acid sequence encoding a polypeptide are known in the art and include isolation from genomic DNA, preparation from cDNA, or a combination thereof. The cloning of the nucleic acid sequences of the present invention from such genomic DNA can be effected, e.g., by using methods based on polymerase chain reaction (PCR) or antibody screening of expression libraries to detect cloned DNA fragments with shared structural features (See, e.g., Innis et al., 1990, PCR: A Guide to Methods and Application, Academic Press, New York.). Other nucleic acid amplification procedures such as ligase chain reaction (LCR), ligated activated transcription (LAT) and nucleic acid sequence-based amplification (NASBA) may be used.

The sequence information as provided herein should not be so narrowly construed as to require inclusion of erroneously identified bases. The specific sequences disclosed herein can be readily used to isolate the complete gene from filamentous fungi, in particular A. niger which in turn can easily be subjected to further sequence analyses thereby identifying sequencing errors.

Unless otherwise indicated, all nucleotide sequences determined by sequencing a DNA molecule herein were determined using an automated DNA sequencer and all amino acid sequences of polypeptides encoded by DNA molecules determined herein were predicted by translation of a nucleic acid sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

The person skilled in the art is capable of identifying such erroneously identified bases and knows how to correct for such errors.

Preferred species of the Aspergillus genus are the filamentous fungi belonging to the Aspergillus niger group, the Aspergillus glaucus group, the Aspergillus terreus group, the Aspergillus restrictus group, the Aspergillus fumigatus group, the Aspergillus cervinus group, the Aspergillus ornatus group, the Aspergillus clavatus group, the Aspergillus versicolor group, the Aspergillus ustus group, the Aspergillus wentii group, the Aspergillus ochraceus group, the Aspergillus candidus group, the Aspergillus cremeus group, the Aspergillus sparsus group, the Aspergillus sojae group, and the Aspergillus oryzae group.

In a second aspect, the invention relates to nucleic acid constructs comprising a DNA sequence according to the first aspect of the invention.

“Nucleic acid construct” is defined herein as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term expression cassette when the nucleic acid construct contains all the control sequences required for expression of a coding sequence. The term “coding sequence” as defined herein is a sequence, which is transcribed into mRNA and translated into a polypeptide comprising acetamidase activity of the present invention. The boundaries of the coding sequence are generally determined by the ATG start codon at the 5′ end of the mRNA and a translation stop codon sequence terminating the open reading frame at the 3′ end of the mRNA. A coding sequence can include, but is not limited to, DNA, cDNA, and recombinant nucleic acid sequences.

Expression will be understood to include any step involved in the production of the polypeptide including, but not limited to transcription, post-transcriptional modification, translation, post-translational modification, and secretion.

The term “control sequences” is defined herein to include all components, which are necessary or advantageous for the expression of a polypeptide. Each control sequence may be native or foreign to the nucleic acid sequence encoding the polypeptide. Such control sequences include, but are not limited to, a leader, optimal translation initiation sequences (as described in Kozak, 1991, J. Biol. Chem. 266:19867-19870), a polyadenylation sequence, a pro-peptide sequence, a pre-pro-peptide sequence, a promoter, a signal sequence, and a transcription terminator. At a minimum, the control sequences include a promoter, and transcriptional and translational stop signals.

The control sequences may be provided with linkers for the purpose of introducing specific restriction sites facilitating ligation of the control sequences with the coding region of the nucleic acid sequence encoding a polypeptide. The term “operably linked” is defined herein as a configuration in which a control sequence is appropriately placed at a position relative to the coding sequence of the DNA sequence such that the control sequence directs the production of a polypeptide.

The control sequence may be an appropriate promoter sequence, a nucleic acid sequence, which is recognized by a host cell for expression of the nucleic acid sequence. The promoter sequence contains transcriptional control sequences, which mediate the expression of the polypeptide. The promoter may be any nucleic acid sequence, which shows transcriptional activity in the cell including mutant, truncated, and hybrid promoters, and may be obtained from genes encoding extracellular or intracellular polypeptides either homologous or heterologous to the cell.

The control sequence may also be a suitable transcription terminator sequence, a sequence recognized by a fungal host cell to terminate transcription. The terminator sequence is operably linked to the 3′ terminus of the nucleic acid sequence encoding the polypeptide. Any terminator, which is functional in the cell, may be used in the present invention.

Preferred terminators for fungal host cells are obtained from the genes encoding A. oryzae TAKA amylase, A. niger glucoamylase (glaA), A. nidulans anthranilate synthase, A. niger alpha-glucosidase, trpC gene and Fusarium oxysporum trypsin-like protease.

The control sequence may also be a suitable leader sequence, a non-translated region of an mRNA, which is important for translation by the fungal host cell. The leader sequence is operably linked to the 5′ terminus of the nucleic acid sequence encoding the polypeptide. Any leader sequence, which is functional in the cell, may be used in the present invention.

Preferred leaders for fungal host cells are obtained from the genes encoding A. oryzae TAKA amylase and A. nidulans triose phosphate isomerase and A. niger glaA.

Other control sequences may be isolated from the Penicillium IPNS gene, or pcbC gene, the beta tubulin gene. All the control sequences cited in WO 01/21779 are herewith incorporated by reference.

The control sequence may also be a polyadenylation sequence, a sequence which is operably linked to the 3′ terminus of the nucleic acid sequence and which, when transcribed, is recognized by the fungal host cell as a signal to add polyadenosine residues to transcribed mRNA. Any polyadenylation sequence, which is functional in the cell, may be used in the present invention.

Preferred polyadenylation sequences for fungal host cells are obtained from the genes encoding A. oryzae TAKA amylase, A. niger glucoamylase, A. nidulans anthranilate synthase, Fusarium oxyporum trypsin-like protease and A. niger alpha-glucosidase.

Manipulation of the nucleic acid sequence encoding a polypeptide prior to its insertion into a vector may be desirable or necessary depending on the expression vector. The techniques for modifying nucleic acid sequences utilizing cloning methods are well known in the art.

In a preferred embodiment, the nucleic acid construct comprising a DNA sequence according to the first aspect of the invention comprises a promoter, which is native to said DNA sequence.

In another preferred embodiment, the nucleic acid construct comprising a DNA sequence according to the first aspect of the invention comprises a promoter, which is foreign to said DNA sequence. In this embodiment, the native promoter of the homologous amdS gene has been replaced by a different promoter. This replacement promoter, which is referred to as foreign promoter herein, can either be stronger than the native amdS promoter or it can be regulated in a different manner. Either way, the replacement of the native amdS promoter is intended to facilitate the selection of transformants, e.g. by increasing the growth advantage of transformants over non-transformants when grown on acetamide or related amide-compounds as sole N- or C-source. Preferably, the foreign promoters are also homologous to the host in which they are used. Suitable foreign promoters can be derived from genes encoding glycolytic enzymes or enzymes involved in alcohol metabolism, such as the promoters from genes encoding phosphoglycerate kinases, glyceraldehyde-phosphate dehydrogenases, triose-phosphate kinases, pyruvate kinase or alcohol dehydrogenases. Examples of preferred inducible promoters that can be used are starch-, copper-, oleic acid-inducible promoters.

In yet another embodiment of the invention, the nucleic acid construct of the previous paragraphs further comprises a gene of interest to be expressed. The gene of interest may be operably linked to separate control sequences or may be under control of the sequences operably linked to the acetamidase gene of the invention.

Preferably, the nucleic acid construct will be comprised in a suitable vector to enable introduction into a host cell. The vector may be any vector (e.g., a plasmid or virus), which can be conveniently subjected to recombinant DNA procedures and can bring about the expression of the nucleic acid sequence encoding the polypeptide. The choice of the vector will typically depend on the compatibility of the vector with the fungal host cell into which the vector is to be introduced. The vectors may be linear or closed circular plasmids. The vector may be an autonomously replicating vector, i.e., a vector, which exists as an extrachromosomal entity, the replication of which is independent of chromosomal replication, e.g., a plasmid, an extrachromosomal element, a minichromosome, or an artificial chromosome. An autonomously maintained doning vector suitable for a fungal host cell may comprise the AMA1-sequence (see e.g. Aleksenko and Clutterbuck (1997), Fungal Genet. Biol. 21: 373-397).

Alternatively, the vector may be one which, when introduced into the fungal host cell, is integrated into the genome and replicated together with the chromosome (s) into which it has been integrated. The integrative cloning vector may integrate at random or at a predetermined target locus in the chromosomes of the fungal host cell. The integrative cloning vector may comprise a DNA fragment, which is homologous to a DNA sequence in a predetermined target locus in the genome of the fungal host cell for targeting the integration of the cloning vector to this predetermined locus. In order to promote targeted integration, the cloning vector is preferably linearized prior to transformation of the host cell. Linearization is preferably performed such that at least one but preferably either end of the cloning vector is flanked by sequences homologous to the target locus. The length of the homologous sequences flanking the target locus is preferably at least 30 bp, preferably at least 50 bp, preferably at least 0.1 kb, even preferably at least 0.2 kb, more preferably at least 0.5 kb, even more preferably at least 1 kb, most preferably at least 2 kb. Preferably, the DNA sequence in the cloning vector, which is homologous to the target locus is derived from a highly expressed locus meaning that it is derived from a gene, which is capable of high expression level in the fungal host cell. A gene capable of high expression level, i.e. a highly expressed gene, is herein defined as a gene whose mRNA can make up at least 0.5% (w/w) of the total cellular mRNA, e.g. under induced conditions, or alternatively, a gene whose gene product can make up at least 1% (w/w) of the total cellular protein, or, in case of a secreted gene product, can be secreted to a level of at least 0.1 g/l (as described in EP 357 127 B1). A number of preferred highly expressed genes of a fungal host cell are given by way of example: the amylase, glucoamylase, alcohol dehydrogenase, xylanase, glyceraldehyde-phosphate dehydrogenase or cellobiohydrolase (cbh) genes from Aspergilli or Trichoderma. Most preferred highly expressed genes for these purposes are a glucoamylase gene, preferably an A. niger glucoamylase gene, an A. oryzae TAKA-amylase gene, an A. nidulans gpdA gene, a Trichoderma reesei cbh gene, preferably cbh1.

The vector system may be a single vector or plasmid or two or more vectors or plasmids, which together contain the total DNA to be introduced into the genome of the filamentous fungal cell, or a transposon.

In a third aspect, the invention relates to a polypeptide having acetamidase activity that is encoded by the DNA sequence of the first aspect of the invention.

In a preferred embodiment, said polypeptide comprises any one of sequences SEQ ID NO: 3, SEQ ID NO: 8, SEQ ID NO: 13, SEQ ID NO: 16, or SEQ ID NO: 19.

In a fourth aspect, the invention relates to a fungal host cell comprising a nucleic acid construct according to the second aspect of the invention.

In a preferred embodiment, said host cell further comprises a gene of interest to be expressed.

The gene of interest to be expressed may encode a polypeptide. The polypeptide may be any polypeptide whether native or heterologous to the fungal host cell. The term “heterologous polypeptide” is defined herein as a polypeptide, which is not produced by a wild-type fungal host cell. The term “polypeptide” is not meant herein to refer to a specific length of the encoded produce and therefore encompasses peptides, oligopeptides and proteins. The polypeptide may also be a recombinant polypeptide, which is a polypeptide native to a cell, which is encoded by a nucleic acid sequence, which comprises one or more control sequences, foreign to the nucleic acid sequence, which is involved in the production of the polypeptide. The polypeptide may be a wild-type polypeptide or a variant thereof. The polypeptide may also be a hybrid polypeptide, which contains a combination of partial or complete polypeptide sequences obtained from at least two different polypeptides where one or more of the polypeptides may be heterologous to the cell. Polypeptides further include naturally occurring allelic and engineered variations of the above-mentioned polypeptides.

Preferably, the polypeptide is an antibody or portions thereof, an antigen, a clotting factor, an enzyme, a hormone or a hormone variant, a receptor or portions thereof, a regulatory protein, a structural protein, a reporter, or a transport protein, intracellular protein, protein involved in secretion process, protein involved in folding process, chaperone, peptide amino acid transporter, glycosylation factor, transcription factor. Preferably, the polypeptide is secreted extracellularly.

Alternatively, the polypeptide is an oxidoreductase, transferase, hydrolase, lyase, isomerase, ligase, catalase, cellulase, chitinase, cutinase, deoxyribonuclease, dextranase, esterase.

Alternatively, the polypeptide is a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. More preferably, the desired gene encodes a phytase. Even more preferably, the polypeptide is an aminopeptidase, amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, proteolytic enzyme, invertase, laccase, lipase, mannosidase, mutanase, oxidase, pectinolytic enzyme, peroxidase, phospholipase, polyphenoloxidase, ribonuclease, transglutaminase, or glucose oxidase, hexose oxidase, monooxygenase.

Alternatively, the polypeptide is human insulin or an analog thereof, human growth hormone, erythropoietin, tissue plasminogen activator (tPA) or insulinotropin.

The nucleic acid sequence encoding a heterologous polypeptide may be obtained from any prokaryotic, eukaryotic, or other source. For purposes of the present invention, the term “obtained from” as used herein in connection with a given source shall mean that the polypeptide is produced by the source or by a cell in which a gene from the source has been inserted.

Alternatively, the polypeptide may be an intracellular protein or enzyme such as for example a chaperone, protease or transcription factor. An example of this is described in Appl Microbiol Biotechnol. 1998 October; 50(4):447-54 (“Analysis of the role of the gene bipA, encoding the major endoplasmic reticulum chaperone protein in the secretion of homologous and heterologous proteins in black Aspergilli. Punt P J, van Gemeren I A, Drint-Kuijvenhoven J, Hessing J G, van Muijlwijk-Harteveld G M, Beijersbergen A, Verrips C T, van den Hondel C A). This can be used for example to improve the efficiency of a host cell as protein producer if this polypeptide, such as a chaperone, protease or transcription factor, was known to be a limiting factor in protein production.

In the methods of the present invention, the fungal host cell may also be used for the recombinant production of polypeptides, which are native to the cell. The native polypeptides may be recombinantly produced by, e.g., placing a gene encoding the polypeptide under the control of a different promoter to enhance expression of the polypeptide, to expedite export of a native polypeptide of interest outside the cell by use of a signal sequence, and to increase the copy number of a gene encoding the polypeptide normally produced by the cell. The present invention also encompasses, within the scope of the term “heterologous polypeptide”, such recombinant production of polypeptides native to the cell, to the extent that such expression involves the use of genetic elements not native to the cell, or use of native elements which have been manipulated to function in a manner that do not normally occur in the filamentous fungal cell. The techniques used to isolate or clone a nucleic acid sequence encoding a heterologous polypeptide are known in the art and include isolation from genomic DNA, preparation from cDNA, or a combination thereof.

In the methods of the present invention, heterologous polypeptides may also include a fused or hybrid polypeptide in which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide.

Techniques for producing fusion polypeptides are known in the art, and include, ligating the coding sequences encoding the polypeptides so that they are in frame and expression of the fused polypeptide is under control of the same promoter (s) and terminator. The hybrid polypeptides may comprise a combination of partial or complete polypeptide sequences obtained from at least two different polypeptides wherein one or more may be heterologous to the mutant fungal cell. An isolated nucleic acid sequence encoding a heterologous polypeptide of interest may be manipulated in a variety of ways to provide for expression of the polypeptide. Expression will be understood to include any step involved in the production of the polypeptide including, but not limited to, transcription, posttranscriptional modification, translation, post-translational modification, and secretion. Manipulation of the nucleic acid sequence encoding a polypeptide prior to its insertion into a vector may be desirable or necessary depending on the expression vector. The techniques for modifying nucleic acid sequences utilizing cloning methods are well known in the art.

In another preferred embodiment, the endogenous acetamidase activity of the fungal host cell is reduced by modification and/or inactivation of the endogenous acetamidase gene or genes by specific or random mutagenesis, site-directed mutagenesis, PCR generated mutagenesis, nucleotide insertion and/or deletion and/or substitution, gene interruption or gene replacement techniques, anti-sense techniques, RNAi techniques, or combinations thereof. Methods include, but are not limited to: subjecting the parent cell to mutagenesis and selecting for mutant cells in which the capability to produce an acetamidase with reduced activity by comparison to the parental cell. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet(W) irradiation, hydroxylamine, N-methyl-N′-nitro-N-nitrosoguanidine (MNNG), 0-methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and selecting for mutant cells exhibiting reduced expression of the gene.

Alternatively, modification or inactivation of the gene may be performed by established anti-sense techniques using a nucleotide sequence complementary to the nucleic acid sequence of the gene. More specifically, expression of the gene by a fungal cell may be reduced or eliminated by introducing a nucleotide sequence complementary to the nucleic acid sequence, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. An example of expressing an antisense-RNA is shown in Appl Environ Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993); 190(2):247-52.).

Furthermore, modification, downregulation or inactivation of the gene may be obtained via the RNA interference (RNAi) technique (FEMS Microb. Lett. 237 (2004): 317-324). In this method identical sense and antisense parts of the nucleotide sequence, which expression is to be affected, are cloned behind each other with a nucleotide spacer in between, and inserted into an expression vector. After such a molecule is transcribed, formation of small (21-23) nucleotide fragments will lead to a targeted degradation of the mRNA, which is to be affected. The elimination of the specific mRNA can be to various extends. The RNA interference techniques described in WO2005/05672A1 and/or WO2005/026356A1 may be used for downregulation, modification or inactivation of the gene.

Alternatively, according to another preferred embodiment of the invention, the sequences of the novel amdS genes are used to inactivate the endogenous copy (or copies) of the amdS gene in the genome of the organism from which the novel amdS gene is derived. To this extent an inactivation vector can be constructed using the sequences of the novel amdS gene to target the vector to an endogenous copy of the gene by homologous recombination. The inactivation can then be caused either by replacement of, or by insertion into the endogenous amdS gene. Inactivation of the endogenous amdS gene provides the advantage of reducing the background of non-transformed cells in transformations using an amdS gene as selectable marker for the introduction of a gene of interest. Alternatively, the endogenous amdS locus can serve as a defined site of integration for genes of interest to be expressed.

In another preferred embodiment, in the fungal host cell transformed with the nucleic acid construct of the second aspect of the invention and optionally comprising a gene of interest to be expressed and/or with reduced endogenous acetamidase activity, the nucleic acid construct of the second aspect of the invention is deleted or rendered inactive by gene replacement and/or inactivation and/or modification and/or disruption of the recombinant DNA construct, or combinations thereof. In this embodiment, transformation of the fungal host cell is followed by subsequent curing of transformants in order to obtain MARKER GENE FREE™ recombinant strains as outlined in EP-A1-O 635 574. Alternatively, curing can be performed using the methods already described for reducing the expression of the endogenous acetamidase genes.

Optionally, the host cell comprises an elevated unfolded protein response (UPR) compared to the wild type cell to enhance production abilities of a polypeptide of interest. UPR may be increased by techniques described in US2004/0186070A1 and/or US2001/0034045A1 and/or WO01/72783A2. More specifically, the protein level of HAC1 and/or IRE1 and/or PTC2 has been modulated in order to obtain a host cell having an elevated UPR.

Alternatively, or in combination with an elevated UPR, the host cell is genetically modified to obtain a phenotype displaying lower protease expression and/or protease secretion compared to the wild-type cell in order to enhance production abilities of a polypeptide of interest. Such phenotype may be obtained by deletion and/or modification and/or inactivation of a transcriptional regulator of expression of proteases. Such a transcriptional regulator is e.g. prtT. Lowering expression of proteases by modulation of prtT may be performed by techniques described in US2004/0191864A1.

Alternatively, or in combination with an elevated UPR and/or a phenotype displaying lower protease expression and/or protease secretion, the host cell displays an oxalate deficient phenotype in order to enhance the yield of production of a polypeptide of interest. An oxalate deficient phenotype may be obtained by techniques described in WO2004/070022A2.

Alternatively, or in combination with an elevated UPR and/or a phenotype displaying lower protease expression and/or protease secretion and/or oxalate deficiency, the host cell displays a combination of phenotypic differences compared to the wild cell to enhance the yield of production of the polypeptide of interest. These differences may include, but are not limited to, lowered expression of glucoamylase and/or neutral alpha-amylase A and/or neutral alpha-amylase B, protease, and oxalic acid hydrolase. Said phenotypic differences displayed by the host cell may be obtained by genetic modification according to the techniques described in US2004/0191864A1.

In a more preferred embodiment, the fungal host cell is a filamentous fungal cell, preferably belonging to a species of the Aspergillus, Penicillium or Trichoderma genus. More preferably, the filamentous fungal host cell belongs to the group of Aspergillus niger, Aspergillus oryzae, Aspergillus sojae, Trichoderma reesei or Penicillium chrysogenum.

In a fifth aspect, the invention relates to a method for the production of a compound of interest in a fungal host cell of the fourth aspect. The host cells of the fourth aspect are cultured under conditions conducive to both the expression of the acetamidase and the compound of interest using methods known in the art. For example, the cells may be cultured by shake flask culture, small-scale or large-scale culture (including continuous, batch, fed-batch, or solid state cultures) in laboratory or industrial fermentors performed in a suitable medium and under conditions allowing the compound of interest to be expressed and/or isolated. The culture takes place in a suitable nutrient medium comprising carbon and nitrogen sources and inorganic salts, using procedures known in the art (see, e.g., Bennett, J. W. and LaSure, L., eds., More Gene Manipulations in Fungi, Academic Press, CA, 1991). Suitable media are available from commercial suppliers or may be prepared using published compositions (e.g., in catalogues of the American Type Culture Collection). Optionally, the compound of interest is recovered from the culture by methods known in the art. If the polypeptide is secreted into the nutrient medium, the polypeptide can be recovered directly from the medium. If the polypeptide is not secreted, it is recovered from cell lysates.

The resulting compound of interest may be isolated by methods known in the art. For example, the compound of interest may be isolated from the nutrient medium by conventional procedures including, but not limited to, centrifugation, filtration, extraction, spray drying, evaporation, or precipitation. The isolated compound of interest may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing, differential solubility (e.g., ammonium sulfate precipitation), or extraction (see, e.g., Protein Purification, J.-C. Janson and Lars Ryden, editors, VCH Publishers, New York, 1989).

In a preferred embodiment, the host cells of the fourth aspect are cultured in a culture medium, wherein the culture medium comprises acetamide as sole carbon and/or nitrogen source, as well as a method wherein said culturing results in the enrichment of the proportion of cells according to invention.

In another preferred embodiment, the background of non-transformants is reduced by adding CsCl to the culture medium comprising acetamide as sole nitrogen and/or carbon source.

The invention further discloses fungal cells according to the invention, preferably filamentous fungal cells, with the ability to grow well on a culture medium containing acetamide as sole carbon and/or nitrogen source and wherein said ability is not caused by the expression of a heterologous acetamidase gene but is rather caused by the expression, preferably over-expression, of a homologous acetamidase gene. The ability of a cell to grow well on a culture medium containing acetamide as sole carbon and/or nitrogen source is herein defined as the ability to grow faster than the corresponding wild-type cell, wherein wild-type is understood to mean wild-type with respect to its acetamidase genotype.

The present invention is further described by the following examples, which should not be construed as limiting the scope of the invention.

### EXAMPLES

### General Molecular Cloning Techniques

In the examples described herein, standard molecular cloning techniques such as isolation and purification of nucleic acids, electrophoresis of nucleic acids, enzymatic modification, cleavage and/or amplification of nucleic acids, transformation of E. coli, etc., were performed as described in the literature (Sambrook et al. (1989) “Molecular Cloning: a laboratory manual”, Cold Spring Harbour Laboratories, Cold Spring Harbour, N.Y.; Innis et al. (eds.) (1990) “PCR protocols, a guide to methods and applications” Academic Press, San Diego). The Aspergillus niger strain (CBS 513.88) used was already deposited at the CBS Institute under the deposit number CBS 513.88.

### Example 1 Identification of Novel A. niger amdS Genes

Novel putative acetamidase genes from A. niger were identified by careful inspection of the full annotated genome sequence of the fungus, through DNA sequence homology searches familiar to those skilled in the art. Acetamidase gene homologue AMD2 is shown as SEQ ID NO: 1, SEQ ID NO: 2 and SEQ ID NO: 3, genomic, cDNA and protein sequence, respectively. Acetamidase gene homologue AAE1 is shown as SEQ ID NO: 6, SEQ ID NO: 7 and SEQ ID NO: 8, genomic, cDNA and protein sequence, respectively. Acetamidase gene homologue AAE2 is shown as SEQ ID NO: 11, SEQ ID NO: 12 and SEQ ID NO: 13, genomic, cDNA and protein sequence, respectively. Acetamidase gene homologue AAE3 is shown as SEQ ID NO: 14, SEQ ID NO: 15 and SEQ ID NO: 16, genomic, cDNA and protein sequence, respectively. Acetamidase gene homologue AAE4 is shown as SEQ ID NO: 17, SEQ ID NO: 18 and SEQ ID NO: 19, genomic, cDNA and protein sequence, respectively.

### Example 2 Molecular Cloning of Novel A. niger amdS Genes

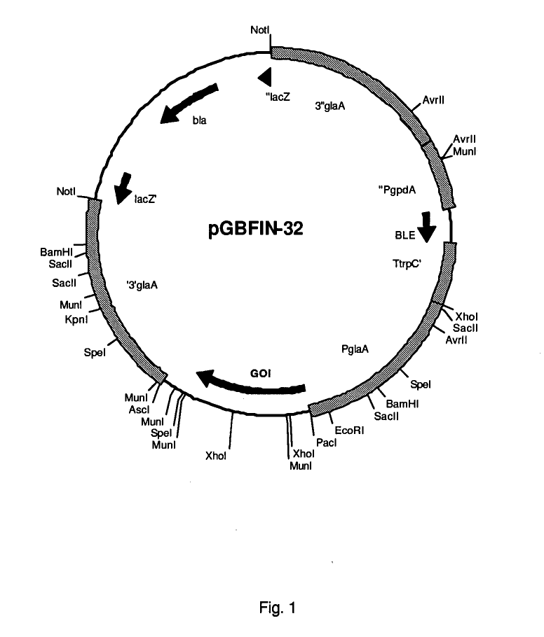

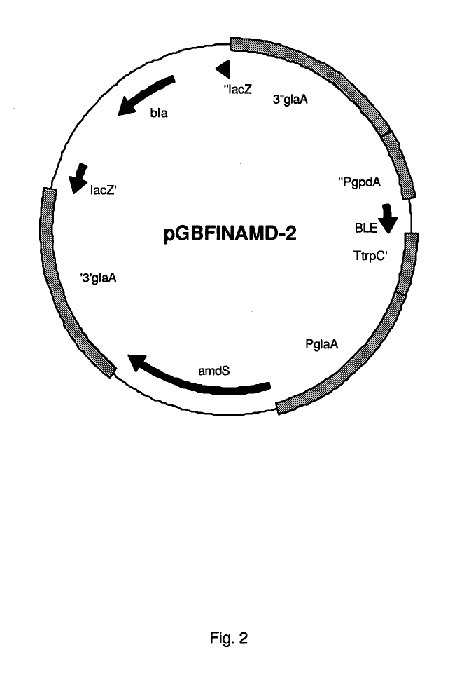

Genomic DNA from CBS513.88 was used as template in a PCR reaction using SEQ ID NO: 4 and SEQ ID NO: 5 to amplify the AMD-2 gene. The resulting PCR fragment (SEQ ID NO: 20) was digested with restriction enzymes PacI and AscI according to the manufacturers instructions and ligated into a PacI, AscI linearised A. niger expression vector as depicted in FIG. 1. This resulted in a construct in which the AMD2 gene encoding a putative A. niger acetamidase was placed under control of the glaA promoter (FIG. 2).

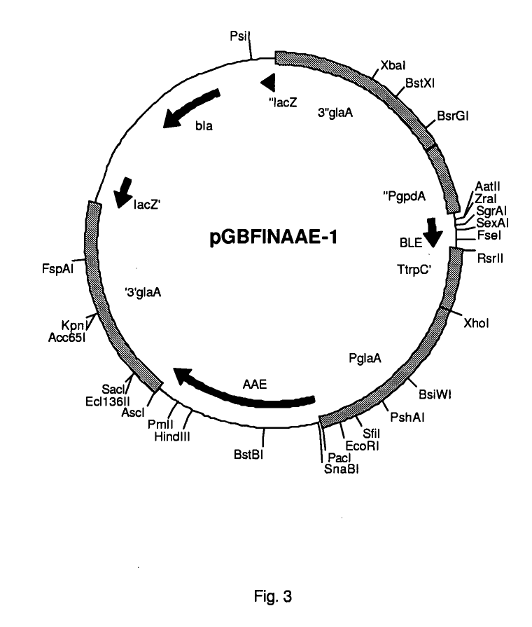

In addition, genomic DNA from CBS513.88 was used as template in a PCR reaction using SEQ ID NO: 9 and SEQ ID NO: 10 to amplify the AAE1 gene. The resulting PCR fragment (SEQ ID NO: 21) was digested with restriction enzymes Pac and AscI according to the manufacturers instructions and ligated into a PacI, AscI linearised A. niger expression vector as depicted in FIG. 1. This resulted in a construct in which the AAE1 gene encoding a putative A. niger acetamidase was placed under control of the glaA promoter (FIG. 3).

The resulting expression constructs (FIGS. 2 and 3) encoding putative novel A. niger acetamidases were used to transform the A. niger host CBS513.88. Transformants were analysed by PCR for the presence of the acetamidase constructs, Selected clones were further analysed in example 3.

### Example 3 Use of Novel A. niger amdS Genes as Selection Marker Genes in Transformation of A. niger CBS 513.88

In order to determine whether genes encoding putative A. niger homologues of acetamidase, could be used as selection marker genes in transformations of A. niger, AMD2 and AAE1 expression constructs (FIGS. 2 and 3) were used to transform A. niger CBS 513.88. Transformants were initially grown on agar medium containing 50 μg/ml phleomycin, to select for transformants containing intact expression constructs, since the plasmid backbone contains the BLE-gene conferring phleomycin resistance (FIGS. 1, 2 and 3). Subsequently, phleomycin-resistant transformants were transferred to agar medium containing acetamide as a sole nitrogen source. Only phleomycin resistant transformants, containing the AMD2 or AAE1 expression constructs (as exemplified by PCR analysis) were able to grow on media containing acetamide as sole nitrogen source, indicating that AMD2 and AAE1 can indeed be used as novel acetamidase marker genes in transformations of A. niger.

The invention described and claimed herein is not to be limited in scope by the specific embodiments herein enclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In case of conflict, the present disclosure including definitions will control.

1. An isolated nucleic acid encoding an acetamidase derivable from an Aspergillus, wherein the nucleic acid has a nucleotide sequence which is not SEQ ID NO: 18, but is selected from the group consisting of:
(a) SEQ ID NO: 1, SEQ ID NO: 6, SEQ ID NO: 11, SEQ ID NO: 14, or SEQ ID NO: 17, and
(b) fragments or mutants of (a).

2. The nucleic acid encoding an acetamidase according to claim 1, wherein said acetamidase comprises an amino acid sequence, wherein the sequence identity of the amino acid sequence with SEQ ID NO: 3, SEQ ID NO: 8, SEQ ID NO: 13, SEQ ID NO: 16, or SEQ ID NO: 19 is more than 40%.

3. A recombinant nucleic acid construct comprising the nucleic acid according to claim 1.

4. The nucleic acid construct according to claim 3, comprising a promoter, which is native to the nucleotide sequence.

5. The nucleic acid construct according to claim 3, comprising a promoter, which is foreign to the nucleotide sequence.

6. The nucleic acid construct according to claim 3 further comprising a gene of interest to be expressed.

7. A polypeptide encoded by the nucleic acid according to claim 1.

8. The polypeptide according to claim 7 having an amino acid sequence comprising SEQ ID NO: 3, SEQ ID NO: 8, SEQ ID NO: 13, SEQ ID NO: 16, or SEQ ID NO: 19.

9. A fungal host cell comprising the nucleic acid construct according to claim 3.

10. A fungal host cell comprising the nucleic construct according to claim 3 and further comprising a gene of interest to be expressed.

11. The fungal host cell according to claim 9 wherein endogenous acetamidase activity is reduced by modification and/or inactivation of the endogenous acetamidase gene or genes by specific or random mutagenesis, site-directed mutagenesis, PCR generated mutagenesis, nucleotide insertion and/or deletion and/or substitution, gene interruption or gene replacement techniques, anti-sense techniques, RNAi techniques, or combinations thereof.

12. The fungal host cell according to claim 9 wherein the nucleic acid, or the nucleic acid construct is deleted or rendered inactive by gene replacement and/or inactivation and/or modification and/or disruption, or combinations thereof.

13. A fungal host cell according to claim 9, wherein the fungal cell is a filamentous fungal cell.

14. A fungal host cell according to claim 13, wherein the filamentous fungal cell is Aspergillus niger, Aspergillus oryzae, Aspergillus sojae, Trichoderma reesei or Penicillium chrysogenum.

15. A method for producing a compound of interest in a fungal host comprising:
a. culturing the fungal host cell of claim 9 under conditions conducive to both expression of the acetamidase and the compound of interest and optionally,
b. recovering the compound of interest.

16. The method according to claim 15, wherein the culture medium contains acetamide as sole carbon and/or nitrogen source.

17. The nucleic acid encoding an acetamidase according to claim 1, wherein the Aspergillus is Aspergillus niger.

18. The nucleic acid encoding an acetamidase according to claim 1, wherein the nucleotide sequence is SEQ ID NO: 1, SEQ ID NO: 6, SEQ ID NO: 11, SEQ ID NO: 14, or SEQ ID NO: 17.

19. The nucleic acid encoding an acetamidase according to claim 2, wherein the sequence identity is at least 90%.

20. A fungal host cell according to claim 13, wherein the filamentous fungal cell is a species of Aspergillus, Penicillium or Trichoderma.
